(19) United States
(12) Patent Application Publication
(10) Pub. No.: US 2011/0071464 A1
(43) Pub. Date: Mar. 24, 2011

(54) SEMI-CLOSED LOOP INSULIN DELIVERY

(75) Inventor: Cesar C. Palerm, Pasadena, CA (US)

(73) Assignee: Medtronic Minimed, Inc., Northridge, CA (US)

(21) Appl. No.: 12/565,574

(22) Filed: Sep. 23, 2009

(51) Int. Cl. A61M 5/168; G08B 23/00

(52) U.S. Cl. 604/66; 340/573.1

(57) ABSTRACT

Subject matter disclosed herein relates to a semi-closed loop drug delivery system. In particular embodiments, an amount of insulin to be administered to a patient may be calculated based, at least in part, on one or more blood-glucose measurements obtained from said patient, and an alarm may be initiated in response to the calculated amount of insulin. At least a portion of the calculated amount of insulin may then be injected into the patient in the absence of a response to the alarm within a time limit.

### BACKGROUND

1. Field

Subject matter disclosed herein relates to a semi-closed loop drug delivery system.

2. Information

The pancreas of a normal healthy person produces and releases insulin into the blood stream in response to elevated blood plasma glucose levels. Beta cells, which reside in the pancreas, produce and secrete insulin into the blood stream as it is needed. If beta cells become incapacitated or die, a condition known as Type 1 diabetes mellitus may result. Also, if beta cells produce insufficient quantities of insulin, Type 2 diabetes may result. In such cases, insulin must be provided to the body from another source.

Traditionally, since insulin cannot be taken orally, insulin has been injected with a syringe. More recently, use of infusion pump therapy has been increasing, especially for delivering insulin to patients. For example, external infusion pumps may be worn on a belt, in a pocket, or the like, and deliver insulin into the body via an infusion tube with a percutaneous needle or a cannula placed in the subcutaneous tissue.

### BRIEF DESCRIPTION OF THE FIGURES

Non-limiting and non-exhaustive embodiments will be described with reference to the following figures, wherein like reference numerals refer to like parts throughout the various figures unless otherwise specified.

FIG. 1 is a perspective view of an embodiment of an infusion device.

FIG. 2 is a schematic block diagram of an infusion device, according to an embodiment.

FIG. 3 is a flow diagram of an infusion device process, according to an embodiment.

FIG. 4 is a flow diagram of an infusion device process, according to another embodiment.

FIG. 5 shows example graphs of blood-glucose and bolus values as a function of time, according to an embodiment.

FIG. 6 shows example graphs of blood-glucose and bolus values as a function of time, according to another embodiment.

### SUMMARY

One or more embodiments described herein relate to a system, method and/or apparatus for calculating an amount of insulin to be administered to a patient based, at least in part, on one or more measurements obtained from the patient; optionally initiating an alarm in response to the calculated amount of insulin; and automatically initiating injection of at least a portion of the calculated amount in the absence of a response to the optional alarm within a time limit of the initiation of said alarm. In one particular implementation, the at least a portion of the calculated amount is less than the calculated amount. In another particular implementation, the amount is calculated by estimating an amount of insulin on board. In yet another particular implementation, sensor measurements may be correlated with a blood-glucose concentration in a patient. In another implementation, sensor measurements may comprise blood-glucose sensor measurements and/or may comprise ketone sensor measurements.

In another particular implementation, the amount of insulin is calculated based, at least in part, on a blood-glucose target and an insulin correction factor associated with the patient. For example, at least a portion of the calculated amount of insulin may be based, at least in part, on a lower limit for a corrected blood-glucose concentration in the patient. In another example, one or more blood-glucose measurements may be taken from a blood glucose sensor, wherein at least a portion of the calculated amount of insulin is based, at least in part, on an estimate of a measurement error associated with the blood-glucose sensor.

In another implementation, a device may comprise at least one sensor to measure blood-glucose concentration of a patient; an optional alarm; an infusion device to deliver fluid to a patient; and one or more processors programmed with instructions to: calculate an amount of fluid to be administered to the patient based, at least in part, on one or more blood-glucose sensor measurements obtained from the patient; optionally initiate activation of the alarm in response to the calculated amount of fluid; and automatically initiate injection of at least a portion of said calculated amount through said infusion device in the absence of a response to said alarm within a time limit of said initiation of said alarm or in the case with no alarm. In one particular implementation, the at least a portion of the calculated amount is less than the calculated amount. In another particular implementation, the one or more processors are further programmed with said instructions to calculate said amount of fluid by estimating an amount of fluid on board. In yet another particular implementation, the fluid comprises insulin.

In yet another particular implementation, the one or more processors are further programmed with instructions to calculate the amount of fluid based, at least in part, on a blood-glucose target and a fluid correction factor associated with the patient. In one particular example, at least a portion of said calculated amount of fluid is based, at least in part, on a lower limit for a corrected blood-glucose concentration in said patient. In another particular example, the one or more blood-glucose measurements are taken from a blood glucose sensor, wherein at least a portion of the calculated amount of fluid is based, at least in part, on an estimate of a measurement error associated with the blood glucose sensor.

In another implementation, an article comprises a storage medium comprising machine-readable instructions stored thereon which, in response to being executed by a processor, enable the processor to: calculate an amount of fluid to be administered to a patient based, at least in part, on one or more blood-glucose sensor measurements obtained from the patient; optionally initiate activation of an alarm in response to the calculated amount of fluid; and automatically initiate injection of at least a portion of the calculated amount into the patient in the absence of a response to the alarm within a time limit of the initiation of said alarm or in the case with no alarm. In a particular implementation, the instructions, in response to being executed by the processor, further enable the processor to calculate the amount of fluid by estimating an amount of fluid on board.

In another particular implementation, the instructions, in response to being executed by the processor, further enable the processor to calculate the amount of fluid based, at least in part, on a blood-glucose target and a fluid correction factor associated with the patient. For example, the at least a portion of the calculated amount of fluid may be based, at least in part, on a lower limit for a corrected blood-glucose concentration in said patient.

One or more additional embodiments described herein relate to a system, method and/or apparatus for measuring a patient's blood-glucose concentration based, at least in part, on measurements obtained from a sensor; calculating a correction bolus based, at least in part, on the measured blood-glucose concentration and the patient's insulin correction factor; calculating a worst-case value of blood-glucose based, at least in part, on the measured blood-glucose concentration and a relative sensor error of said sensor; calculating a maximum allowable bolus based, at least in part, on the worst-case value of blood-glucose concentration and a safety target limit; and delivering less than the correction bolus to the patient if said correction bolus is less than the maximum allowable bolus. In one particular implementation, a bolus delivered to the patient may be further reduced based, at least in part, on an amount of insulin on-board.

In another particular implementation, a device comprises at least one sensor to measure blood-glucose concentration of a patient; and one or more processors programmed with instructions to: calculate a correction bolus based, at least in part, on said measured blood-glucose concentration and said patient's insulin correction factor; calculate a worst-case value of blood-glucose concentration based, at least in part, on said measured blood-glucose concentration and a relative sensor error of said sensor; calculate a maximum allowable bolus based, at least in part, on said worst-case value of blood-glucose concentration and a safety target limit; and initiate delivery of less than said correction bolus to said patient if said correction bolus is less than said maximum allowable bolus. In a particular implementation, the one or more processors are further programmed with instructions to further reduce a bolus delivered to said patient based, at least in part, on insulin-on-board.

It should be understood, however, that the above described embodiments are merely directed to example implementations, and that claimed subject matter is not limited to these particular implementations.

### DETAILED DESCRIPTION

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments.

Though continuous subcutaneous insulin infusion (CSII) therapy provides benefits to diabetic patients, ketoacidosis (DKA) is a problematic condition that may strike such patients for a number of reasons. For example, DKA may be observed in clinical practice if a patient's plasma glucose levels are 250 mg/dl or higher, with a concentration of ketone bodies in the blood starting to rise even at lower glucose levels. Such patients may have hyperglycemia due to underestimated carbohydrate content in meals, insulin resistance due to illness (which can appear even before other symptoms are apparent), as well as missed meal boluses, for example.

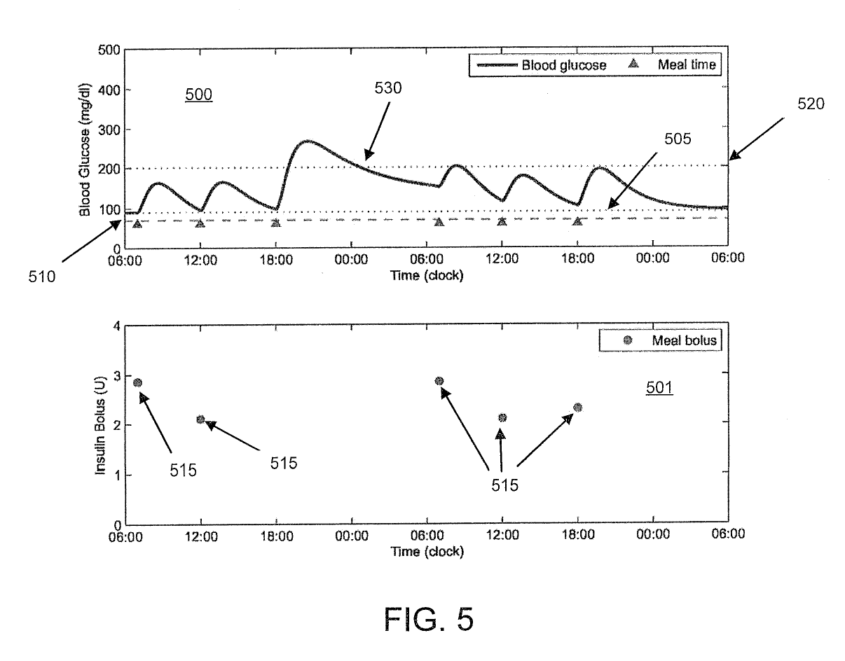

In one particular example, for the purpose of illustration, FIG. 5 shows plots of blood-glucose and bolus values as a function of time, according to an embodiment. Here, such blood-glucose measurements may be obtained using any one of several techniques such as, for example, processing signals provided by a blood-glucose sensor as described below. Additionally, bolus values may be calculated based, at least in part, on processed blood-glucose measurements and/or an insulin correction factor (I_{CF}). In the particular situation represented by the graphs, produced via computer simulation, a patient may have an optimal basal insulin infusion rate of 0.95 U/h to maintain a blood glucose level of 90 mg/dl. The patient's optimal insulin correction factor may be 46 mg/dl per 1 U of insulin (i.e., I_{CF}=46). Simulations that resulted in the graphs were run for 48 h, starting at 6:00 on the first day. Three meals were given at 7:00, 12:00, and 18:00 on both days, for which a meal insulin bolus is optimized.

Continuing with FIG. 5, the top graph 500 comprises a plot of blood-glucose versus time, with the lower dotted line 505 showing basal blood-glucose of 90 mg/dl and the upper dotted line 520 showing a threshold level for the simulation at 200 mg/dl. The lower dashed line 510 is at 70 mg/dl in order to indicate a threshold for hypoglycemia, for example. Triangles indicate times for meals. The lower graph 501 comprises a plot showing a dose of each insulin bolus. Circles 515 denote meal boluses. As shown in FIG. 5, a missed meal-insulin-bolus at 18:00 hours on the first day may result in a simulated patient's blood-glucose levels going above 200 mg/dl. Such levels 530 remain elevated throughout the night and into the next day.

In an embodiment, a semi-closed loop technique may be incorporated in a system that includes an insulin pump and a blood-glucose sensor, which may automatically measure a patient's blood-glucose continually, for example. Such a system may administer insulin correction boluses in order to prevent severe hyperglycemia and therefore also prevent DKA.

In an embodiment, a partial insulin correction bolus to be administered to a patient may be calculated based, at least in part, on one or more blood-glucose measurements obtained automatically by a sensor with or without action by the patient. Such a partial correction bolus may be administered in order to prevent severe hyperglycemia and therefore also prevent DKA, therefore improving overall glycemic control.

In one particular embodiment, if one or more blood-glucose measurements are such that a calculated amount of insulin is beyond a threshold value, then an audio, vibrational/mechanical, and/or visual alarm or other notification directed to a patient may be activated, though such an alarm or notification is optional. Subsequently, an injection of insulin may be initiated by the notified patient. However, if the patient fails to respond to such an alarm within a particular amount of time, or time limit, at least a portion of the calculated amount of insulin may be automatically injected into the patient. In an embodiment where such an alarm or notification is not implemented, failure of a patient to manually inject insulin within a particular amount of time from when blood-glucose measurements surpassed a threshold level may initiate an automatic insulin injection into the patient. Such a process of monitoring blood-glucose levels and insulin delivery to a patient may be performed by an infusion system, according to a particular implementation. Such an infusion system may include at least one sensor to monitor blood-glucose concentration of a patient and an infusion device for delivering fluid, such as insulin, to the patient. Such a sensor may produce at least one sensor signal used by an infusion device to determine a patient's present and/or future blood-glucose levels. Of course, such a process and infusion system are merely examples, and claimed subject matter is not so limited. For example, one or more measurements of a patient other than blood-glucose measurements may be performed, and a variety of other fluids may be substituted for insulin in the descriptions above. In addition, some embodiments may be employed in various infusion environments including, but not limited to a biological implant environment. Other environments may include, but are not limited to, external infusion devices, pumps, and so on. Fluids that may be infused include, but are not limited to, insulin formulations and other formulations having other pharmacological properties, for example.

In one embodiment, an infusion device may deliver fluid, such as insulin, to a patient if future blood-glucose levels are in a patient's predefined target range. In another embodiment, an infusion device may suspend and resume fluid delivery based, at least in part, on future blood-glucose levels and a patient's predefined low shutoff threshold, for example. In still another embodiment, an infusion device may suspend fluid delivery if a future blood-glucose level falls below a predefined low shutoff threshold. In still another embodiment, an infusion device may resume fluid delivery if a future blood-glucose level is above such a predefined low shutoff threshold.

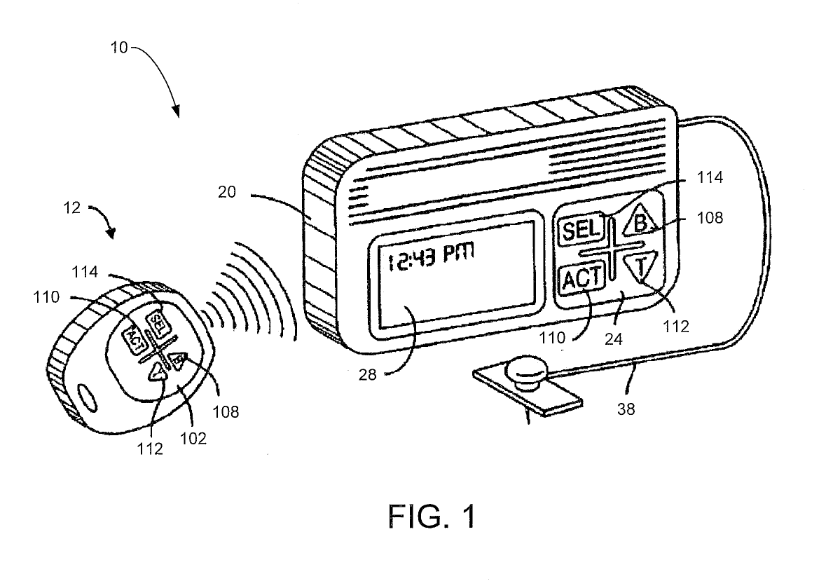

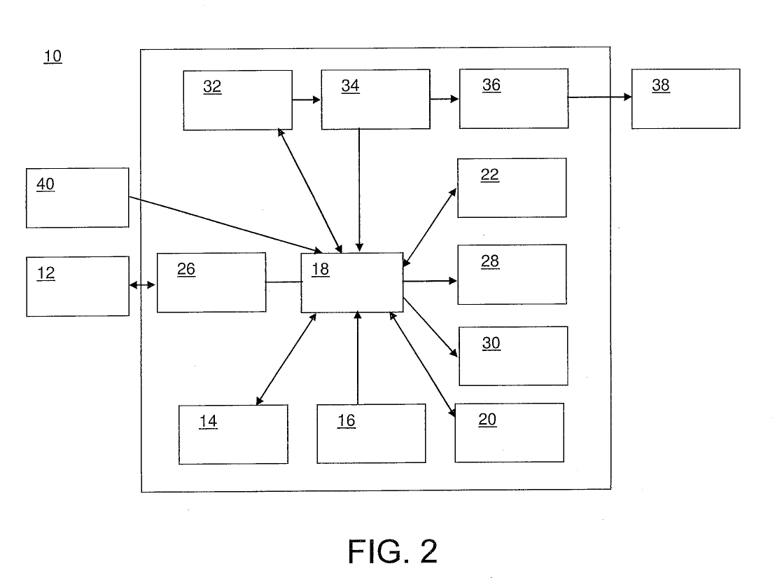

FIG. 1 is a perspective view of an infusion device 10 and FIG. 2 is a schematic block diagram of such an infusion device, according to a particular embodiment. Infusion device 10 may include an optional remote RF programmer 12, a bolus capability 14, and/or an alarm 16. RF programmer 12 and bolus capability 14 may communicate with a processor 18 contained in a housing 20 of infusion device 10. Processor 18 may be used to run programs and/or control infusion device 10, and may be connected to an internal memory device 22 that stores programs, historical data, and/or user defined information and parameters. In a particular embodiment, infusion device 10 may comprise an external infusion pump that is programmed through a keypad 24 on housing 20 or by commands received from RF programmer 12 via a transmitter/receiver 26. Feedback from infusion device 10 on status and/or programming changes may be displayed on an LCD 28 and/or audibly through a speaker 30. In alternative embodiments, the keypad 24 may be omitted and the LCD 28 may be used as a touch screen input device or the keypad 24 may utilize more keys or different key arrangements then those illustrated in the figures. Processor 18 may also be coupled to a drive mechanism 32 that is connected to a fluid reservoir 34 containing fluid that is expelled through an outlet 36 in reservoir 34 and housing 20, and then into a body of a user through tubing and a hypodermic set 38. In other alternative embodiments, keypad 24, LCD 20, and/or speaker 24 may be omitted from infusion device 10, and programming and/or data transfer may be handled through RF programmer 12.

In a particular implementation, infusion device 10 may comprise an external insulin pump having a capability to deliver 0 to 35 Units/hour in basal rates and up to 25.0 Units per meal bolus of U-100 Insulin. Of course, such an implementation is described merely as an example, and claimed subject matter is not so limited. For instance, an external pump may deliver other concentrations of insulin, or other fluids, and may use other limits on a delivery rate.

To deliver a bolus, a user may operate keypad 24 and keys 108, 110, 112 and/or 114 to program and/or deliver one or more bolus types through a single touch key or by the use of one or more menus. In an alternative embodiment, a user may program and/or deliver a bolus via optional RF programmer 12. Such a bolus may comprise a fluid such as medication, chemicals, enzymes, antigens, hormones, and/or vitamins, for example, into a body of a user. In a particular embodiment, infusion device 10 may comprise an external infusion pump, which includes an RF programming capability, a blood-glucose estimation capability, and/or vibration alarm capability. Particular embodiments may be directed towards use in humans; however, in alternative embodiments, external infusion devices may be used in non-human animals.

In a particular embodiment, a sensor 40 included in infusion device 10 may be implanted in and/or through subcutaneous, dermal, sub-dermal, inter-peritoneal, and/or peritoneal tissue. In other particular embodiments, a sensor and/or monitor may be used to determine glucose levels in the blood and/or body fluids of a user without the use or necessity of a wire or cable connection between a transmitter and monitor. However, in still other embodiments, a sensor and/or monitor may be used to determine levels of other agents, characteristics or compositions, such as hormones, cholesterol, medication concentrations, pH, oxygen saturation, viral loads (e.g., HIV), and/or the like. Such a sensor may also include a capability to be programmed and/or calibrated using data received by a telemetered characteristic monitor transmitter device, and/or may be calibrated at a monitor device (or receiver). Such a telemetered characteristic monitor system may be used for applications involving subcutaneous human tissue. However, other applications may involve other types of human or animal tissue, such as muscle, lymph, organ tissue, veins, arteries, and/or or the like. Sensor readings may be provided intermittently or continually. Of course, such details of sensors are merely examples, and claimed subject matter is not so limited.

In a particular embodiment, one or more bolus estimation algorithms may render bolus recommendations based, at least in part, upon various parameters including, but not limited to meal content, blood glucose concentrations, blood glucose concentration time rate of change, insulin-on-board, insulin duration factor, target blood glucose, and/or insulin sensitivity, just to name a few examples. In a particular implementation, again referring to FIG. 2, various parameters may be entered by a user, provided to processor 18 by sensor 40, and/or downloaded from a remote computer, just to name a few examples.

In an embodiment, a bolus estimation algorithm may provide bolus recommendations based, at least in part, upon meal content (user input), blood-glucose concentration (BG) (user and/or meter input), and/or blood glucose concentration time rate of change. In particular implementations, such blood-glucose concentration and/or blood-glucose concentration rate of change may be derived from data furnished by one or more sensors such as a continuous ketone sensor or a continuous glucose sensor and/or monitoring system, or any other sensor capable of providing measurements which are correlated with blood-glucose concentration in the patient. Here, such a sensor may be implanted in the patient or otherwise be brought in to contact with patient tissue or fluids, for example. Meal content may be calculated by the user and entered directly into an infusion device. In another embodiment, meal content may be downloaded from a remote computer containing a food library or the like. In yet another embodiment, a user's blood-glucose concentration may be directly entered into a processor of an infusion device by a glucose meter with or without patient interaction. In still another embodiment, a user's BG concentration rate of change may be received by a processor directly from an external and/or implantable continuous glucose monitoring system, for example. Sensor estimated glucose concentration (SG) may be determined by a calibrated glucose sensor system included in an infusion device. Of course, such details of a bolus estimation algorithm are merely examples, and claimed subject matter is not so limited.

In another embodiment, an infusion device may receive information from various linked devices including, but not limited to a continuous glucose monitoring system, a glucose meter, and/or a remote computer, just to name a few examples. An infusion device may receive information in five-minute intervals, for example, from any one or more of such linked devices. In a particular implementation, receive-time may range from about 1.0 to 10.0 minutes, and information may be received in 20, 30, 40, 50 or 60 minute intervals. Of course, such values are mentioned here as merely examples, and claimed subject matter is not so limited.

In another embodiment, a derivative predicted algorithm may be utilized by an infusion device to compute proportional blood-glucose correction if measured blood-glucose values are outside of a patient's target range. In a particular implementation, such a derivative predicted algorithm may also make correction adjustments for insulin-on-board values and/or compute food corrections. A derivative predicted algorithm may utilize BG information gathered from the patient, glucose monitor, glucose meter, and/or continuous glucose monitoring system, just to name a few examples. In another particular implementation, a processor employing a derivative predicted algorithm may receive data from a continuous and/or near continuous glucose monitoring system where automatic measurements may be taken over a specified period of time.

In an embodiment, sensor-derived blood-glucose levels may be based, at least in part, on trends yielding a prediction of blood-glucose levels at a given number of minutes into the future. Future BG values may be obtained and/or predicted by using a derivative of a current BG value as described by a derivative predicted algorithm. Such blood-glucose levels are termed “derivative corrected” blood glucose levels. To determine derivative corrected blood glucose, various processes or algorithms may be employed utilizing patient-defined parameters, sensor readings, and/or infusion device defined parameters, for example. In a particular implementation, particular processes or algorithms may accept continuous glucose sensor input and use blood-glucose data to make correction adjustments based, at least in part, upon the derivative of sensor derived blood-glucose values.

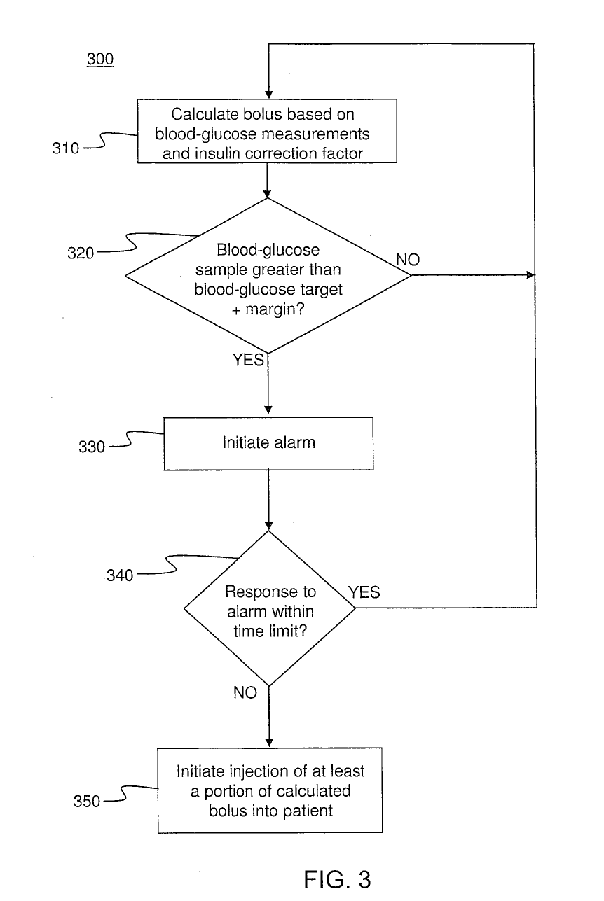

FIG. 3 is a flow diagram of an infusion device process 300, according to an embodiment. A semi-closed loop infusion device, such as infusion device 10 described above, may provide alarm-based capabilities. For example, such a device may calculate a delivery dosage to determine whether to initiate an alarm as a result of estimated blood-glucose in a patient. In another example, such a device may perform delivery dosage calculations to determine whether to initiate an alarm as a result of measured blood-glucose in a patient. In detail, at block 310, a bolus and/or a temporary increase in the basal rate may be calculated based, at least in part, on blood-glucose measurements and an insulin correction factor associated with a particular patient. Such a calculation may also determine a time period for which such a temporary increase in the basal rate is to be applied, for example. At block 320, a determination may be made as to whether an estimate of blood-glucose concentration is greater than a blood-glucose target value. In one particular implementation, if one or more blood-glucose measurements are less than a blood-glucose target value, then process 300 may return to block 310, where blood-glucose measurements may automatically continue. On the other hand, if blood-glucose measurements exceed a blood-glucose target value (plus margin, if any), then process 300 may proceed to block 330, where an infusion device may initiate an alarm. In another particular embodiment, process 300 may proceed to block 330 if blood-glucose measurements exceed a particular margin above a blood-glucose target value. Such a margin may be determined so that if a patient's blood-glucose is substantially over a blood-glucose target value by the margin, then severe hyperglycemia and potentially DKA may occur unless additional insulin is administered.

At block 340, if a patient fails to respond to the alarm within a time limit, then process 300 may proceed to block 350, where an infusion device may initiate an injection of at least a portion of the bolus calculated in block 310. On the other hand, if a patient responds to the alarm within a time limit, then process 300 may proceed to return to block 310, where blood-glucose measurements may automatically continue without injection of bolus.

In another embodiment, process 300 may be extended to include generating an alarm to indicate a potential problem with an infusion site of a bolus injection. For example, an infusion site failure may occur because a cannula infusing insulin is not properly delivering the insulin and/or injury/damage to the tissue may prevent the insulin from being absorbed by the body. In such a case, an insulin pump's back-pressure alarm may not trigger even though insulin is not being absorbed by the patient's body. Accordingly, glucose levels may start to rise. If a patient's glucose levels do not decrease even during insulin bolus delivery, then a failed infusion site may be a source of such a problem. In such a case, an alarm condition may be generated to alert a patient to change their infusion set.

Alarms of an infusion device may include, but are not limited to audible alarms, vibration alarms, and/or visual alarms, just to name a few examples. Additional embodiments may include one type of alarm or a combination of various alarms. Further embodiments may allow a patient to configure which type of alarm is used. For example, such embodiments may allow a patient to set a particular type of alarm to indicate that a bolus has been calculated and is ready to be administered, while another type of alarm may indicate that measured blood-glucose has fallen below a threshold. Alternatively, all alarms may be set the same. A patient may also program the intensity of alarms. Audible alarms may have the capability to increase and/or decrease in volume, change tones, provide melodies, and the like. Vibration alarms may change in intensity and/or pulse to provide tactile alerts. Visual alarms may come in many forms including, but not limited to flashing LCD backlights, and/or flashing LEDs, for example. Response to such alarms may include depressing a button, touching at least a portion of a touch screen, and/or speaking a particular command, just to name a few examples.

In other embodiments, an infusion device may initiate an alarm, such as at block 330 based, at least in part, on sensor-detected readings and/or sensor-derived trends. For example, in an insulin based infusion system for a diabetic patient, if a sensor detects a low blood-glucose level (i.e. hypoglycemia) over a designated period of sensor readings, an infusion device may initiate an alarm and/or stop insulin delivery unless the patient responds to such an alarm within a particular time limit.

In an embodiment, an infusion device, such as infusion device 10 shown in FIG. 1, may provide an automatic insulin correction bolus if a sensor glucose level (G_{S}) reaches a threshold value (Gₜₕ). Such an infusion device may then calculate, using a patient's correction factor, an insulin bolus dose to bring glucose levels to a target blood glucose (G_{T}). In a particular implementation, an infusion device may maintain a condition Gₜₕ≧G_{T}+20 mg/dl to avoid delivering negligible calculated amounts of insulin. An amount of insulin to deliver may be calculated in a similar manner as a patient may normally do by using the patients' insulin correction factor I_{CF}, which is defined as the total mg/dl drop in blood glucose resulting from one unit of insulin bolus. Accordingly,

B=(G_{S}−G_{T})/I_{CF}

where B is the amount of a correction bolus, which can be adjusted based on insulin on board (IOB).

For example, a threshold glucose level may be set to be 200 mg/dl, since at such a level ketone body concentrations may start to rise in blood and in general would be undesirable glucose levels. A target glucose level may be set at 180 mg/dl, which is an upper limit (postprandial peak) for blood glucose as recommended by the American Diabetes Association (2008) standard of care position statement. While such values may be reasonable, they can be adjusted to, for example, have a target blood glucose of 130 mg/dl, which is an upper limit recommended by the American Diabetes Association (2008) for the preprandial periods.

Therefore, with an automatic bolus triggering at 200 mg/dl, and a target glucose level of 180 mg/di, and assuming a typical correction factor of 50 mg/dl/U, we have

$$B = ( 200 - 180 ) / 50 = 0.4 U .$$

If there is insulin on board, it is conceivable that an infusion device may adjust an amount to zero insulin. In such a case, blood glucose may have the potential to continue to rise. In one implementation, a technique to avoid such a situation may comprise initiating an additional blood-glucose measurement at a future point in time, say 30 minutes later (among several other options). Accordingly, there may be two possibilities at this later time: either a sensor glucose level drops below 200 mg/dl, in which case nothing else need be done, or the sensor glucose level remains above 200 mg/dl. If the glucose level remains above 200 mg/dl, then an infusion device may deliver a new bolus if the rate of change of glucose level is greater than, say, −1 mg/dl/min (e.g., glucose levels are stable or rising). This situation may be common, for example in the case of a missed meal bolus.

According to an embodiment, accuracy of sensor measurements may be considered in providing an infusion device that operates safely for patients. For example, a lower limit on a target blood glucose may be established so that an automatic correction to a target of 70-110 mg/dl is not permitted by a infusion device. In another example, an insulin dose may be limited by a infusion device based, at least in part, on a worst-case scenario that considers a relative error of one or more sensors on the infusion device.

To illustrate a particular example, a bolus calculated at block 310 in FIG. 3 may be adjusted based, at least in part, on a relative absolute deviation, or error E, of a sensor. A value for E may be considered to be around 16%, though a higher value may be used, as is shown below. For a given sensor glucose sample G_{S} compared to a reference measurement G_{B}, the relative absolute deviation may be given by

B=|[(200−180)/50]|.

Then, for a given value of E (e.g., 0.16 for the 16% case), possible values of blood glucose level may be calculated. Thus

G_{B}=G_{S}/(1+E)

In one implementation, as indicated above, a more conservative approach may comprise using twice an assumed relative error, so that a worst case value of blood glucose G_{Bwc} may be given by

G_{Bwc}=G_{S}/(1+2E)

Considering a safety target limit G_{Tsl}, below which an insulin correction may be avoided, and again using a patient's correction factor I_{CF}, a infusion pump may calculate an insulin bolus that would bring the patient to G_{Tsl}, which may be used as a constraint for a maximum allowable bolus dose Bmax. In such a case,

Bmax=(G_{Bwc}−G_{Tsl})/I_{CF}.

Accordingly, Bmax may comprise a maximum allowable bolus to be administered at block 350 in FIG. 3.

To illustrate an example, consider that sensor glucose is G_{S}=300 mg/dl, with a target of G_{T}=180 mg/dl and a correction factor of I_{CF}=50 mg/dl/U. Then the a calculated correction bolus may be

$$B = ( 300 - 180 ) / 50 = 2.4 U .$$

A safety target limit of G_{Tsl}=100 mg/dl and a relative error of 15% (E=0.15) may result in

$${G}_{Bwc} = 300 / ( 1 + 2 ( 0.15 ) ) = 231 mg \text{/} dl$$

$$and$$

$$Bmax = ( 231 - 100 ) / 50 = 2.6 U .$$

Accordingly, in this case, since B<Bmax, a full correction bolus may be safely applied, since even assuming a 30% relative error in sensor glucose measurements, a infusion device may avoid blood-glucose levels below 100 mg/dl. If the assumed relative error were 20% then Bmax=2 U, which may comprise a maximum amount of insulin that could safely be delivered. In another implementation, IOB may be considered in determining Bmax. Also, other constraints may be imposed, such as withholding additional corrections based, at least in part, on whether a bolus was given in the immediate past hour or two, for example.

In one embodiment, relative error may be determined in real-time for a particular sensor that a patient may be wearing. Such a determination may be performed by using a recursive weighted average, in which an initial value may be assumed to be known (and may be based, at least in part, on known statistics from sensor trials). Then, if a patient takes a fingerstick measurement (be it used for calibration or not), a relative error for that one point may be calculated and be used to correct and/or adjust a value for E. For example, if a particular sensor is not performing well, a value of E may increase, leading to a safety mechanism of an infusion device becoming more conservative. On the other hand, if a sensor is performing well, safety constraints may be relaxed, although for safety reasons such constraints may still be capped so that an assumed relative error does not go below a certain threshold.

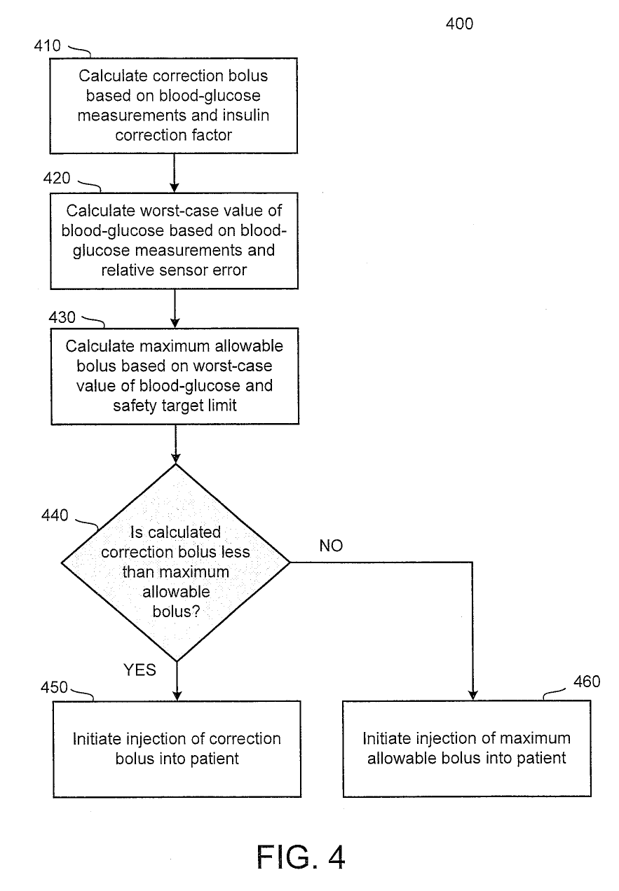

FIG. 4 is a flow diagram of an infusion device process 400, according to another embodiment. At block 410, as described above, a correction bolus may be calculated based, at least in part, on measured blood-glucose and a patient's insulin correction factor. At block 420, a worst-case value of blood-glucose may be calculated based, at least in part, on blood-glucose measurements and a margin of error that may result from errors introduced by a blood-glucose sensor of an infusion device. Such sensor errors may comprise, for example, a sensor bias and/or sensor measurement noise. At block 430, a maximum allowable bolus may be calculated based, at least in part, on a worst-case value of blood-glucose and a safety target limit, as indicated above. At block 440, a determination is made whether a calculated correction bolus is less than a maximum allowable bolus. If such a calculated correction bolus is less than a maximum allowable bolus, then process 400 may proceed to block 450, where an infusion device may deliver a full correction bolus, as calculated at block 410, to a patient. On the other hand, if a determination is made that a calculated correction bolus is greater than a maximum allowable bolus, then process 400 may proceed to block 460, where an infusion device may deliver less than a full correction bolus. Instead, merely a maximum allowable bolus, as calculated at block 430, may be delivered to a patient.

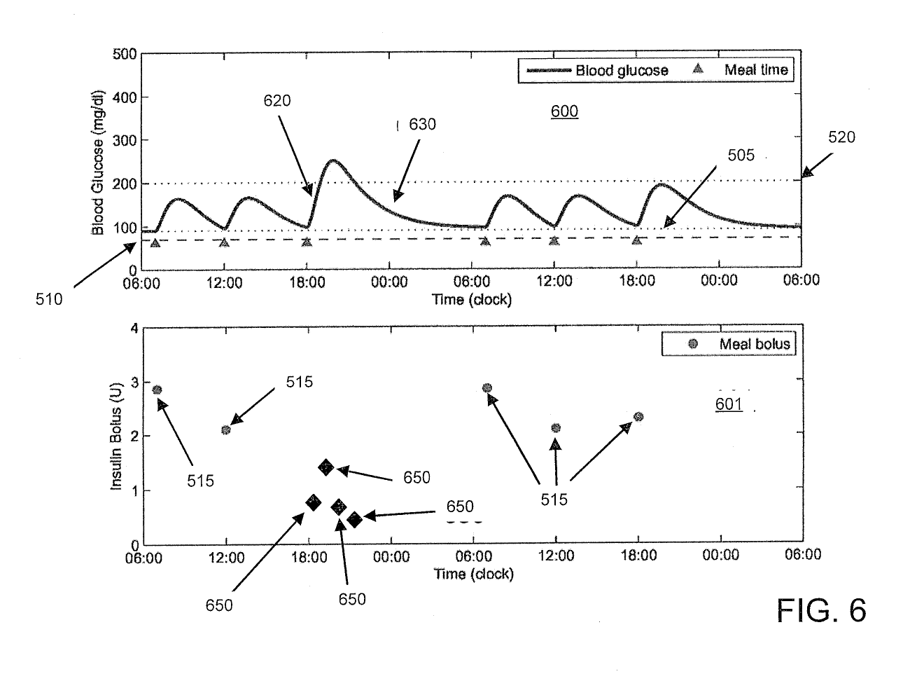

FIG. 6 shows example graphs of blood-glucose and bolus values as a function of time, according to another embodiment. Simulation values used for the case shown in FIG. 5 were repeated for the case represented by FIG. 6, except that a process, such as process 400 for example, was applied for the case represented by FIG. 6. Accordingly, a series of boluses 650, shown in lower graph 601, are delivered to the simulated patient in response to an excessive increase 620 in the patient's blood-glucose values, resulting from a missed meal-insulin-bolus at 18:00 hours. Such boluses 650 may be calculated at block 310 in process 300 and administered to the patient at block 350, as described above for FIG. 3, for example. As shown in the upper graph 600, boluses 650 result in an accelerated decrease 630 in the patient's blood-glucose values relative the rate of decrease 530, shown in FIG. 5. Thus, although blood glucose levels rise more than in an ideal case wherein a meal bolus is given correctly (at 18:00 hours on the second day), glucose levels do stabilize and are almost back to normal during an overnight period.

A notable situation may occur if a patient's insulin sensitivity decreases by a relatively large portion. Such a situation may occur during illness (e.g., the flu) and/or with certain drugs used to treat other conditions. Such drugs, including Prednisone for example, may induce insulin resistance. In such cases, it is not uncommon for insulin requirements to double. Even so, a bolus estimation algorithm may render bolus recommendations based, at least in part, upon blood glucose concentrations responsive to such a change in insulin sensitivity.

In the above detailed description, numerous specific details are set forth to provide a thorough understanding of claimed subject matter. However, it will be understood by those skilled in the art that claimed subject matter may be practiced without these specific details. In other instances, methods, apparatuses, or systems that would be known by one of ordinary skill have not been described in detail so as not to obscure claimed subject matter.

Some portions of the detailed description above are presented in terms of algorithms or symbolic representations of operations on binary digital signals stored within a memory of a specific apparatus or special purpose computing device or platform. In the context of this particular specification, the term specific apparatus or the like includes a general purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a specific apparatus, such as a special purpose computer or a similar special purpose electronic computing device. In one example, such a special purpose computer or special purpose electronic computing device may comprise a general purpose computer programmed with instructions to perform one or more specific functions. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

The terms, “and,” “and/or,” and “or” as used herein may include a variety of meanings that will depend at least in part upon the context in which it is used. Typically, “and/or” as well as “or” if used to associate a list, such as A, B or C, is intended to mean A, B, and C, here used in the inclusive sense, as well as A, B or C, here used in the exclusive sense. Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments. Embodiments described herein may include machines, devices, engines, or apparatuses that operate using digital signals. Such signals may comprise electronic signals, optical signals, electromagnetic signals, or any form of energy that provides information between locations.

While there has been illustrated and described what are presently considered to be example embodiments, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular embodiments disclosed, but that such claimed subject matter may also include all embodiments falling within the scope of the appended claims, and equivalents thereof.

1. A method comprising:
calculating an amount of insulin to be administered to a patient based, at least in part, on sensor measurements obtained from said patient;
initiating an alarm in response to said calculated amount of insulin; and
automatically initiating injection of at least a portion of said calculated amount in the absence of a response to said alarm within a time limit of said initiation of said alarm.

2. The method of claim 1, wherein said at least a portion of said calculated amount is less than said calculated amount.

3. The method of claim 1, wherein said calculating said amount of insulin comprises estimating an amount of insulin on board.

4. The method of claim 1, wherein said calculating said amount of insulin further comprises calculating said amount of insulin based, at least in part, on a blood-glucose target and an insulin correction factor associated with said patient.

5. The method of claim 4, wherein said at least a portion of said calculated amount of insulin is based, at least in part, on a lower limit for a corrected blood-glucose concentration in said patient.

6. The method of claim 1, wherein said sensor measurements are correlated with a blood-glucose concentration in said patient.

7. The method of claim 1, wherein said sensor measurements comprise blood-glucose sensor measurements.

8. The method of claim 1, wherein said sensor measurements comprise ketone sensor measurements.

9. The method of claim 4, wherein said blood-glucose concentration is measured by a blood glucose sensor, and wherein said at least a portion of said calculated amount of insulin is based, at least in part, on an estimate of a measurement error associated with said blood glucose sensor.

10. A device comprising:
at least one sensor to measure blood-glucose concentration of a patient;
an alarm;
one or more processors programmed with instructions to:
calculate an amount of fluid to be administered to said patient based, at least in part, on sensor measurements obtained from said patient;
initiate said alarm in response to said calculated amount of fluid; and
automatically initiate injection of at least a portion of said calculated amount in the absence of a response to said alarm within a time limit of said initiation of said alarm; and
an infusion device to deliver said amount of fluid to said patient

11. The device of claim 10, wherein said at least a portion of said calculated amount is less than said calculated amount.

12. The device of claim 10, wherein said one or more processors are further programmed with said instructions to calculate said amount of fluid by estimating an amount of fluid on board.

13. The device of claim 10, wherein said one or more processors are further programmed with instructions to calculate said amount of fluid by calculating said amount of fluid based, at least in part, on a blood-glucose target and a fluid correction factor associated with said patient.

14. The device of claim 13, wherein said at least a portion of said calculated amount of fluid is based, at least in part, on a lower limit for a corrected blood-glucose concentration in said patient.

15. The device of claim 10, wherein said sensor measurements are correlated with a blood-glucose concentration in said patient.

16. The device of claim 10, wherein said sensor measurements comprise blood-glucose sensor measurements.

17. The device of claim 10, wherein said sensor measurements comprise ketone sensor measurements.

18. The device of claim 12, wherein said blood-glucose concentration is measured by a blood glucose sensor, and wherein said at least a portion of said calculated amount of fluid is based, at least in part, on an estimate of a measurement error associated with said blood glucose sensor.

19. The device of claim 10, wherein said fluid comprises insulin.

20. An apparatus comprising:
means for calculating an amount of insulin to be administered to a patient based, at least in part, on measurements obtained from said patient;
means for initiating an alarm in response to said calculated amount of insulin; and
means for automatically initiating injection of at least a portion of said calculated amount in the absence of a response to said alarm within a time limit of said initiation of said alarm.

21. The apparatus of claim 20, wherein said at least a portion of said calculated amount is less than said calculated amount.

22. The apparatus of claim 20, wherein said means for calculating said amount of insulin comprises means for estimating an amount of insulin on board.

23. The apparatus of claim 20, wherein said means for calculating said amount of insulin further comprises means for calculating said amount of insulin based, at least in part, on a blood-glucose target and an insulin correction factor associated with said patient.

24. The apparatus of claim 23, wherein said at least a portion of said calculated amount of insulin is based, at least in part, on a lower limit for a corrected blood-glucose concentration in said patient.

25. The apparatus of claim 20, wherein said sensor measurements are correlated with a blood-glucose concentration in said patient.

26. The apparatus of claim 20, wherein said sensor measurements comprise blood-glucose sensor measurements.

27. The apparatus of claim 20, wherein said sensor measurements comprise ketone sensor measurements.

28. The apparatus of claim 23, wherein said blood-glucose concentration is measured by a blood glucose sensor, and wherein said at least a portion of said calculated amount of insulin is based, at least in part, on an estimate of a measurement error associated with said blood glucose sensor.

29. An article comprising:
a storage medium comprising machine-readable instructions stored thereon which, in response to being executed by a processor, enable said processor to:
calculate an amount of insulin to be administered to a patient based, at least in part, on sensor measurements obtained from said patient;
initiate activation of an alarm in response to said calculated amount of insulin; and
automatically initiate injection of at least a portion of said calculated amount into said patient in the absence of a response to said alarm within a time limit of said initiation of said alarm.

30. The article of claim 29, wherein said instructions, in response to being executed by said processor, further enable said processor to calculate said amount of insulin by estimating an amount of insulin on board.

31. The article of claim 29, wherein said instructions, in response to being executed by said processor, further enable said processor to calculate said amount of insulin by calculating said amount of insulin based, at least in part, on a blood-glucose target and a insulin correction factor associated with said patient.

32. The article of claim 31, wherein said at least a portion of said calculated amount of insulin is based, at least in part, on a lower limit for a corrected blood-glucose concentration in said patient.

33. A method comprising:
measuring a patient's blood-glucose concentration based, at least in part, on measurements obtained at a sensor;
calculating a correction bolus based, at least in part, on said measured blood-glucose concentration and said patient's insulin correction factor.
calculating a worst-case value of blood-glucose based, at least in part, on said measured blood-glucose concentration and a relative sensor error of said sensor;
calculating a maximum allowable bolus based, at least in part, on said worst-case value of blood-glucose concentration and a safety target limit; and
delivering less than said correction bolus to said patient if said correction bolus is less than said maximum allowable bolus.

34. The method of claim 33, further comprising:
further reducing a bolus delivered to said patient based, at least in part, on insulin-on-board.

35. A device comprising:
at least one sensor to measure blood-glucose concentration of a patient;
one or more processors programmed with instructions to:
calculate a correction bolus based, at least in part, on said measured blood-glucose concentration and said patient's insulin correction factor.
calculate a worst-case value of blood-glucose concentration based, at least in part, on said measured blood-glucose concentration and a relative sensor error of said sensor; and
calculate a maximum allowable bolus based, at least in part, on said worst-case value of blood-glucose concentration and a safety target limit; and
initiate delivery of less than said correction bolus to said patient if said correction bolus is less than said maximum allowable bolus.

36. The device of claim 35, wherein said one or more processors are further programmed with instructions to further reduce a bolus delivered to said patient based, at least in part, on insulin-on-board.

37. A method comprising:
calculating an amount of insulin to be administered to a patient based, at least in part, on measurements obtained from said patient;
automatically initiating injection of at least a portion of said calculated amount if said patient fails to manually inject insulin within a time limit if blood-glucose measurements surpass a threshold level.

38. The method of claim 37, wherein said at least a portion of said calculated amount is less than said calculated amount.

39. The method of claim 37, wherein said calculating said amount of insulin comprises estimating an amount of insulin on board.

40. The method of claim 37, wherein said sensor measurements are correlated with a blood-glucose concentration in said patient.

41. The method of claim 37, wherein said sensor measurements comprise blood-glucose sensor measurements.

42. The method of claim 37, wherein said sensor measurements comprise ketone sensor measurements.
